(19) United States
(12) Patent Application Publication
(10) Pub. No.: US 2024/0400978 A1
(43) Pub. Date: Dec. 5, 2024

(54) Biocompatible Structures for Connecting and Cultivating Biological Material

(71) Applicant: EBERHARD KARLS UNIVERSITAET TUEBINGEN MEDIZINISCHE FAKULTAET, Tübingen (DE)

(72) Inventors: Kevin ACHBERGER, Tübingen (DE); Stefan LIEBAU, Rangendingen (DE)

(73) Assignee: EBERHARD KARLS UNIVERSITAET TUEBINGEN MEDIZINISCHE FAKULTAET, Tübingen (DE)

(21) Appl. No.: 18/733,422

(22) Filed: Jun. 4, 2024

Related U.S. Application Data
(63) Continuation of application No. PCT/EP2022/084841, filed on Dec. 7, 2022.

(30) Foreign Application Priority Data
Dec. 7, 2021 (DE) 102021132190.5

(51) Int. Cl. C12N 5/00; C12N 5/079

(52) U.S. Cl. CPC C12N 5/0068; C12N 5/0062; C12N 5/0622; C12N 2513/00; C12N 2523/00; C12N 2533/90

(57) ABSTRACT

The present invention relates to a method for producing biocompatible structures for the connection and cultivation of biological material, a method for cultivating aggregates of biological material, and the use of biocompatible structures for the connection and cultivation of biological material.

### CROSS-REFERENCES TO RELATED APPLICATIONS

This application is a continuation of copending International patent application PCT/EP2022/084841 filed on 7 Dec. 2022 and designating the United States of America, which was not published in English, and claims priority of German patent application DE 10 2021 132 190.5 filed on 7 Dec. 2021. The entire contents of these prior applications are incorporated herein by reference.

### FIELD OF THE INVENTION

The present invention relates to a method for producing biocompatible structures for connecting and cultivating biological material, a method for cultivating aggregates of biological material, and the use of biocompatible structures for connecting and cultivating biological material.

### BACKGROUND OF THE INVENTION

Traditionally, biological cells are cultured in vitro, i.e., in culture dishes, in essentially three different ways: 1. On coated or uncoated plastic or glass surfaces; the coating is usually done with gels or proteins as well as peptides of the extracellular matrix; culturing on uncoated plastic surfaces is referred to as adherent cell culture; 2. on semipermeable membranes (“Transwell”); and 3. as three-dimensional cultures, such as suspension cultures.

With the first two methods, cells or cell mixtures from (primary) tissues or derived from stem cells can be cultured, which can achieve a certain level of cell maturity and functionality. However, these cells are not very physiological and can rarely reproduce complex cell and tissue interactions.

Suspension cultures of cell aggregates, so-called spheroids or organoids, offer the advantage that the cells, usually self-organizing, can form complex structures and interactions. Spheroids can, for example, be produced through self-aggregation. Here, adherent single or mixed cultures are brought together in a vessel and then independently form spheres or differentiate into organ-like, complex organoids. Examples include retinal organoids, which form a light-sensitive, structured retina. Intestinal organoids can represent intestinal transport functions and contribute to intestinal villi in vivo. Other examples are brain organoids, kidney organoids, etc.

However, most of these organoids or spheroids remain individual organ/tissue systems and do not form complex organ systems with each other. They also generally do not form vascular and ductal structures or structures that grow or migrate into the organ from other body areas during normal development.

One possibility to achieve this to a certain extent is offered by the so-called assembloids. These represent the fusion of several organoids into a morphological and functional unit. An example of this is cortico-spinal-muscle assembloids, which consist of brain regions (cortex), spinal spheroids (spinal), and muscle cells; see Anderson et al., Generation of Functional Human 3D Cortico-Motor-Assembloids. Cell, Volume 183, 2020, pages 1913-1929, e1-10.

However, the assembloids, like the organoids, are difficult to orient and largely fuse randomly with each other. The organoids and assembloids are often of different sizes, making it challenging to spatially align them correctly. Often, one organoid or assembloid “absorbs” and overgrows another. Furthermore, the possibilities to provide organoids and assembloids with a structure are very limited. Organoids and assembloids can only be connected “in series,” but not in other orientations. Finally, there is no simple way to add missing individual cells or substances or functional structures, such as immune cells, to the fused organoids or assembloids.

### SUMMARY OF THE INVENTION

Against this background, the invention aims to provide a method that avoids or at least reduces the disadvantages of the cultivation methods known in the prior art.

The objective underlying the invention is achieved by a method for producing biocompatible structures for connecting and cultivating biological material (“Linkerspheres”), which comprises the following steps:
1) Providing a nonpolar, water-immiscible biocompatible liquid, preferably mineral oil;
2) Introducing a solution of a biocompatible matrix material into the liquid to obtain an emulsion;
3) Incubating the emulsion;
4) Forming a three-dimensional structure from the matrix material in the emulsion through incubation to obtain the Linkerspheres.

According to the invention, “biological material” includes biological cells, aggregates of biological cells, organs, and parts thereof.

According to the invention, the nonpolar, water-immiscible biocompatible liquid provided in step (1) is one that behaves like mineral oil, is 100% immiscible with water to form the emulsion according to the invention, and does not possess any toxic properties for biological material.

Preferably, the liquid provided in step (1) is “mineral oil” (CAS number: 8042-47-5; EC number: 232-455-8), i.e., a highly viscous bioreagent that has a quality and purity level allowing its use in molecular biology, for example, for overlaying aqueous solutions and centrifugation gradients (quality level 200). Suitable mineral oils according to the invention include those from Sigma-Aldrich (M5904), Carl Roth GmbH & Co. KG (#8904), Merck (#107160; #113898). Synonyms for the mineral oil according to the invention are “paraffin oil” and “vaseline oil”. The mineral oil is preferably provided in a container, more preferably in a microreaction vessel of suitable size, such as with a volume of 2 ml, 1.5 ml, 0.5 ml, 0.2 ml, etc. (e.g., “Eppendorf” vessel).

According to the invention, a “matrix material” is understood to be a mixture of molecules that is present in liquid form in step 2 and can be transformed into a solid, tissue-like form through the action of physical or chemical phenomena. In its solid form, the matrix material provides a biocompatible, preferably network-like structure that allows the cultivation of biological material. Examples of suitable matrix materials according to the invention include hydrogels, basement membrane-like matrices (e.g., Matrigel; contains a high favorable proportion of laminin), and other biocompatible, gel-like substances.

The introduction of the solution of the biocompatible matrix material into the mineral oil in step (2) is carried out using a suitable dispensing device, such as a pipette. Due to the fluidity of the aqueous matrix material, it is preferably introduced into the mineral oil in the form of a liquid droplet to form an emulsion.

The size of the Linkerspheres according to the invention can be easily controlled by the volume of the matrix material. A larger volume results in larger Linkerspheres, while a smaller volume results in smaller Linkerspheres.

According to the invention, in step (3), an incubation is carried out for a period that allows the formation of the Linkerspheres, preferably from at least 10 seconds up to 60 minutes.

The exact duration is determined by the skilled person and depends on the type of gelling process. For example, matrigels gel at higher temperatures. Other hydrogels require “crosslinkers” or similar binding chemicals. Thus, the incubation can last from a few seconds to minutes (up to 1 hour).

In step (4) of the method according to the invention, due to interfacial effects between the aqueous biocompatible matrix material and the surrounding mineral oil, the aqueous biocompatible matrix material in the emulsion assumes a spherical shape, which gives the Linkerspheres their name.

The objective underlying the invention is thus completely achieved.

The inventors have recognized that using the method according to the invention, biocompatible structures in the form of Linkerspheres can be created, which represent a valuable tool for tissue engineering, capable of at least partially or even completely overcoming the disadvantages of the prior art.

According to the invention, the Linkerspheres obtained by the method are suitable and configured to cultivate biological material within and/or on them. They are further suitable and configured to connect biological material, acting as a bridge or link between different biological materials or aggregates thereof (“linker”=connection, “spheres”=balls). Since the Linkerspheres can cultivate biological material, it is possible for the biological material or parts thereof to grow through or on the Linkerspheres. Thus, a connection can be established via the Linkerspheres between a first biological material or aggregate thereof and a second biological material or aggregate thereof or further biological materials or aggregates thereof.

The Linkerspheres provide a physiological environment for biological material that also allows the formation of vascular and conduit pathways, which, during the development of an organism, grow into or migrate into an organ from different body regions. The Linkerspheres obtained according to the invention enable the reproduction of complex cell, tissue, and organ interactions. They are thus particularly suitable for studying complex biological structures, such as organs, organ systems, etc.

In one embodiment of the method according to the invention, the matrix material is a basement membrane-like matrix.

According to the invention, a “basement membrane-like matrix” is understood to be a complex mixture of biomolecules used as a growth substrate, i.e., as a matrix or cell substrate, in 3D cell culture and tissue engineering. A basement membrane-like matrix is the purified secretion of the murine sarcoma cell line Engelbreth-Holm-Swarm (EHS cells) and resembles in its composition the extracellular matrix of the basement membranes of animal cells. It contains, among other things, laminin, entactin, collagen, and heparan sulfate proteoglycans. This approach has the advantage of employing a particularly suitable biocompatible matrix material according to the invention. The basement membrane-like matrix forms a networked, gel-like structure or hydrogel at approximately 37° C. through the polymerization of the contained proteins, while it is liquid at lower temperatures, for example, at 4° C. Compared to polylysine-coated cell culture matrices, basement membrane-like matrices lead to the formation of a complex, physiological, three-dimensional cell network structure. An overview of basement membrane-like matrices can be found, for example, in Hughes et al: “A complex protein mixture required for optimal growth of cell culture”, in Proteomics, Volume 10, Issue 9, 2010, ISSN 1615-9861, pp. 1886-1890.

Trade names for basement membrane-like matrices suitable for the invention include matrigel (Corning Life Sciences), BME, EHS matrix, etc.

In one embodiment of the invention, in step (2) of the method, a solution of a growth factor-reduced (GFR) basement membrane-like matrix is introduced.

This approach has the advantage of using a matrix in which growth factors are reduced, which can affect the cells in the aggregate (e.g., EGF). For example, neurons are preferably cultured on such a matrix to reduce this influence. A suitable GFR basement membrane-like matrix according to the invention is marketed under the trade name Corning® Matrigel® Growth Factor Reduced (GFR) Basement Membrane Matrix, LDEV-free, product number 354230, Corning Life Sciences.

In one embodiment of the invention, in step (3) of the method, a treatment of the emulsion to solidify the matrix material is carried out, preferably by heating the emulsion, preferably at approximately 37° C., more preferably for about 15 to 30 minutes.

This approach has the advantage that the Linkerspheres are brought into a manageable and suitable solid form for cultivation purposes through the crosslinking of the matrix material or the contained proteins. By solidifying or crosslinking, a solid hydrogel or gel-like structure is formed, which enables the cultivation of and ingrowth with biological material. The heat treatment can conveniently be performed by placing the reaction vessel containing the emulsion into a water bath.

In a further embodiment of the method according to the invention, the solution of the biocompatible matrix material contains cell culture medium.

This measure creates the physiological conditions necessary for the Linkerspheres to be used for cultivating biological material immediately after their production. Any type of culture medium is suitable according to the invention, with the specific choice made by the specialist depending on the biological material to be cultivated or connected with the Linkerspheres. For example, a cell culture medium suitable for cultivating astrocytes is N2 medium and “B27-based retinal differentiation medium” (BRDM medium).

According to a further embodiment of the invention, the solution of the matrix material contains biological material, preferably biological cells.

This measure integrates the biological material or cells of interest into the Linkerspheres already during their production. Such Linkerspheres containing biological material can be used as independent cultivation units or for connecting units or aggregates of biological material.

In one embodiment of the invention, the solution of the biocompatible matrix material contains a dye.

This measure has the advantage of improving the visibility of the Linkerspheres, thereby facilitating their handling and use in subsequent applications. Suitable dyes are biocompatible dyes, for example, dissolved in buffer.

In one embodiment of the invention, in step (2) of the method, the solution of the matrix material is introduced as fluid droplets into the mineral oil, preferably via a pipette tip.

This measure allows for particularly effective production of the Linkerspheres according to the invention. The pipette, with its tip containing the solution of the biocompatible matrix material, is held directly above the surface of the oil liquid. When using a micropipette, it is preferably pressed to the first stop. This way, a droplet forms at the front of the pipette. By dipping the pipette tip into the oil liquid, the droplet detaches from the tip and sinks into the oil liquid to the bottom of the vessel.

In a further development of the method according to the invention, the following step is carried out after step (4):
5) Isolating the Linkerspheres from the emulsion.

This measure advantageously allows the Linkerspheres to be separated from the emulsion and prepared for further use.

In one embodiment of the method according to the invention, the following step is carried out after step (5):
6) Washing the isolated Linkerspheres, preferably with an aqueous solution, more preferably with cell culture medium.

This measure advantageously removes the remaining mineral oil from the Linkerspheres. This washing step can be repeated to remove all excess oil from the Linkerspheres, preventing any residual oil from causing harm in subsequent cultivation applications.

In another embodiment of the invention, the following additional step is carried out after step (4) and before step (5):
4′) Introducing an aqueous solution, preferably an aqueous buffer solution, into the emulsion to form an aqueous phase and transferring the Linkerspheres into the aqueous phase.

This measure creates a two-phase system with an oily phase and an aqueous phase. The formed aqueous Linkerspheres transition from the oil phase to the aqueous phase, from which they can be easily isolated and purified. Any commercially available biocompatible buffer is generally suitable, such as a PBS buffer. The specific choice of buffer by the specialist depends on the specific biological material to be cultivated or connected.

In another embodiment of the invention, the isolated and possibly washed Linkerspheres are transferred into a culture vessel, preferably a culture dish.

This measure places the Linkerspheres in an environment that allows their direct use for cultivating and connecting biological material. Commercially available culture dishes, such as Petri dishes, are suitable, and their selection depends on the specific use of the Linkerspheres.

In one embodiment of the method according to the invention, the isolated and possibly washed and transferred Linkerspheres are cultured, preferably at approximately 37° C., more preferably at approximately 20% O₂, further preferably at approximately 5% CO₂, and more preferably for at least 12 hours.

This measure has the advantage of utilizing particularly suitable parameters for the cultivation of the Linkerspheres.

Another aspect of the present invention relates to a method for cultivating and/or connecting aggregates of biological material, comprising the following steps:
1) Bringing the aggregates into contact with biocompatible structures for connecting and cultivating biological material (“Linkerspheres”) in a suitable medium to obtain a complex of the aggregates and the Linkerspheres,
2) Cultivating the complexes of the aggregates and the Linkerspheres, wherein the Linkerspheres were obtained according to the manufacturing method of the invention.

The features, properties, developments, and advantages of the manufacturing method according to the invention apply equally to the cultivation method according to the invention.

According to the invention, the “cultivation” of the complexes of the aggregates and the Linkerspheres takes place under standard cell culture conditions, for example, in an incubator in a nutrient medium at approximately 37° C. and, if applicable, 5% CO₂.

Using the Linkerspheres according to the invention, the aggregates of biological material can be connected or fused at a defined distance determined by the Linkerspheres. In one embodiment of the invention, more than one Linkersphere or any number of them can be interposed. The distance created by the Linkerspheres ensures a spatial separation of the aggregates to be fused; thus, they cannot readily grow into each other, preventing one aggregate from overgrowing another. The Linkerspheres also allow the connection of multiple aggregates to the same aggregate to obtain, for example, a cloverleaf-like structure.

By connecting or fusing the aggregates via multiple Linkerspheres, theoretically infinitely large and complex arrangements can be created, similar to a building block system (“Lego system”).

Using the Linkerspheres, pathway systems can be created that connect the aggregates of biological material, such as conductive blood vessels, nerve pathways, or structures of the extracellular matrix (ECM). These can also be combined. The pathway systems can either be provided as additional spheroid structures (e.g., vascular organoids) or embedded in the Linkerspheres during their production. The fusion of multiple Linkerspheres with different cell types is also easily possible.

The Linkerspheres can contain any ECM structures and thus mimic body/pathway structures.

With the Linkerspheres according to the invention, the fusion of aggregates of different sizes is easily possible, as they only need to be coupled to the Linkerspheres, and then pathway systems can be interposed between them.

In one embodiment of the cultivation method according to the invention, the aggregates of biological material are cut before being brought into contact with the Linkerspheres, preferably with micro scissors, and more preferably, the aggregates of biological material are brought into contact with the Linkerspheres with the cut surface.

This measure brings the aggregates into a shape and size that align with the size of the Linkersphere and/or the desired connection. The cut surface creates an especially good contact area and ensures a good connection with the aggregates of biological material.

In one embodiment of the cultivation method according to the invention, the cultivation is carried out at approximately 37° C., preferably at approximately 20% O₂, and more preferably at approximately 5% CO₂.

This measure has the advantage of providing the skilled person with cultivation conditions that have proven particularly effective according to expert knowledge.

In another embodiment of the cultivation method according to the invention, the aggregates of biological material are organoids and/or spheroids and/or assembloids.

This measure has the advantage of reducing or even avoiding the disadvantages described in the prior art for organoids and assembloids. By interposing Linkerspheres, there is no direct fusion of potentially different tissue types, which typically does not occur in the organism. The interposition of the Linkerspheres also allows the correct and arbitrary spatial orientation of the aggregates, which in direct fusion, especially with differently sized aggregates, usually occurs randomly and without direction. Additionally, the method according to the invention allows for the orientation of the aggregates “in series” for the first time.

In one embodiment of the cultivation method according to the invention, the aggregates of biological material are selected from the group consisting of: retinal organoid, vascular organoid, brain organoid, neurospheroid.

This measure applies the cultivation method according to the invention to biological aggregates whose natural counterparts have a complex environment that can now be replicated for the first time by the invention. A “retinal organoid” is a three-dimensional structure of retinal cells, differentiated from pluripotent stem cells, containing layering, cell-cell interactions, and cell type-specific diversity that mimics the human embryonic retina. Furthermore, photoreceptors that are light-sensitive and have the specialized structure (inner and outer segments) of this cell type can form in the retinal organoids. A “vascular organoid” is understood to be a three-dimensional structure that can be formed from pluripotent stem cells and contains cells of the vascular system, including endothelial cells and pericytes. Vascular organoids self-organize into a 3D capillary network surrounded by a basement membrane. A “brain organoid” is an organoid formed from pluripotent stem cells that can reflect the organization and cell diversity of specific brain regions. For example, a thalamic organoid includes neurons found in the thalamus. According to the invention, a “neurospheroid” is understood to be a spheroidal arrangement of neural cells (neurons, glial cells, and their precursors) that can be generated from pluripotent stem cells. This arrangement can include organized (e.g., neuro-rosettes or cortex-like areas) and unorganized regions.

In one embodiment of the cultivation method according to the invention, the aggregates of biological material comprise astrocytes, preferably those derived from induced or embryonic pluripotent stem cells (iPSCs).

This measure has the advantage of utilizing cells that are particularly important for tissue engineering, especially of great interest for neuroscientific studies and drug testing. The stem cells are preferably of human or animal origin.

In one embodiment of the invention, the following step is carried out in the cultivation method after step (2):
(3) Repeating steps (1) and (2).

This measure allows for the creation of tissue constructions of unlimited length and connections between Linkerspheres and aggregates of biological material in any number. According to the invention, steps (2) and (3) can therefore be repeated at least once, twice, three times, four times, . . . ten times, twenty times, a hundred times, etc.

Another aspect of the present invention relates to the use of Linkerspheres, obtained according to the production method of the invention, for connecting and cultivating biological material.

The features, properties, developments, and advantages of the production method according to the invention and the cultivation method according to the invention apply equally to the use according to the invention.

It is understood that the features mentioned above and those to be explained below can be used not only in the specified combinations but also in other combinations or individually, without departing from the scope of the present invention.

The invention is now explained in more detail using examples. The features mentioned there apply not only in relation to the specific example but also in isolated form as belonging to the invention.

Reference is made to the accompanying figures.

### BRIEF DESCRIPTION OF THE FIGURES

FIG. 1: Schematic representation of the production of the Linkerspheres according to the invention;

FIG. 2: Astrolinker in schematic (a) and microscopic representations (b)-(e);

FIG. 3: Schematic representation of the connection process of whole and halved, cut organoids with the Linkerspheres (top), and microscopic representation of the linkage product (bottom);

FIG. 4: Microscopic representation of the process of connecting a retinal organoid and a Linkersphere (a), a suspension culture of the linkage product (b), the outgrowth of axons from the retinal organoid into a Linkersphere (c), the linkage product in a single connection (d) and in a double connection (e);

FIG. 5/6: Microscopic representation of a double connection of a Linkersphere with a Neurosphere and a retinal organoid;

FIG. 7: Microscopic representation of a double connection of an Astrolinker with a Neurosphere and a retinal organoid;

FIG. 8: Schematic representation of a complex optic nerve model using Linkerspheres according to the invention;

FIG. 9: Schematic representation of supporting vascularization with Linkerspheres according to the invention;

FIG. 10A: Shows a multi-linking concept in which vascularized nerve formations are combined via linkerspheres.

FIG. 10B: Shows a multi-linking concept in abstract form, in which the linkerspheres combine multicellular tissues.

### EXAMPLES

### 1. Production of Linkerspheres

### Overview

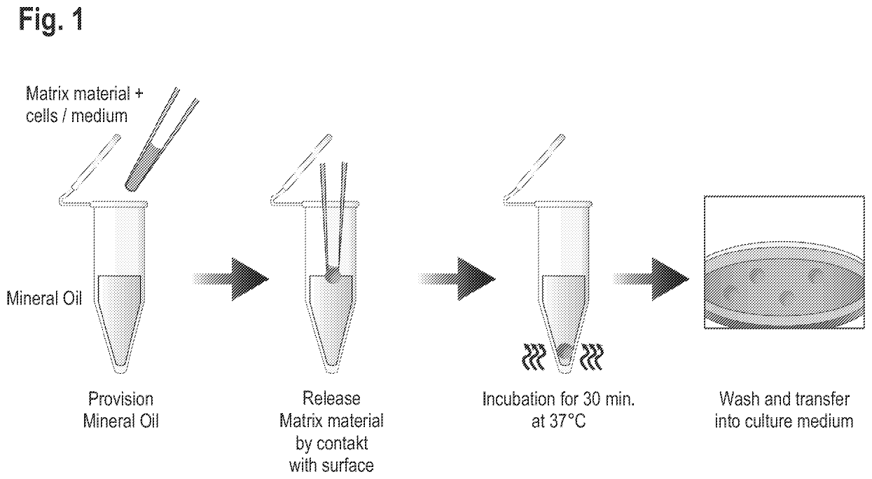

FIG. 1 schematically illustrates the production of Linkerspheres in an overview. In a first step, shown on the far left, mineral oil is provided in a reaction vessel. The liquid, biocompatible matrix material, possibly mixed with biological material such as biological cells and/or culture medium, is drawn into a pipette.

In the next step, a drop of the solution of the biocompatible matrix material is introduced into the mineral oil. For this purpose, the biocompatible matrix material at the bottom of the pipette is brought into contact with the surface of the mineral oil. The drop volume is then expelled from the pipette, allowing the drop to sink to the bottom of the reaction vessel filled with mineral oil.

In the following step, the reaction vessel containing the formed emulsion undergoes heat treatment, for example, by placing it in a water bath and incubating for 30 minutes at 37° C. The proteins of the biocompatible matrix material crosslink, and the matrix material solidifies, forming a gel-like structure or hydrogel, the “Linkersphere.”

In the operation shown as the last step in FIG. 1, the Linkersphere is washed and transferred into a culture medium.

### Details

Linkerspheres with Cells

The following describes the technical details for the production of Linkerspheres containing cells, using the example of Linkerspheres that contain astrocytes, known as “Astrolinkers.”

The following materials are required:
Growth factor-reduced Matrigel (Corning Life Sciences)
Mineral oil (Sigma Aldrich)
N2 Medium (DMEM/F12 with Glutamax, 2% hormone mix, 1% non-essential amino acids (NEAA), 1% antibiotics-antimycotics (Anti-Anti), all Thermo Fisher Scientific)
BRDM Medium (DMEM/F12 (3:1) with Glutamax, 2% B27 without Vitamin A, 1% AA, 1% NEAA, all Thermo Fisher Scientific)
ASC++ Medium (N2 Medium+10 ng/ml epidermal growth factor (EGF)+10 ng/ml fibroblast growth factor 2 (FGF2))
BRDM FBST (DMEM/F12 (3:1) with Glutamax, 10% FBS, 2% B27, 1% AA, 1% NEAA, all Thermo Fisher Scientific, 100 μM taurine)
PBS without magnesium/calcium (PBS−−, Thermo Fisher Scientific)
TrypLE (Thermo Fisher Scientific)
1.5 ml reaction tubes
15 ml conical tubes
Heating block for tubes
Non-adherent 24- or 48-well plates
1000 μl pipette tips with cut-off tips (using scissors)
Water bath at 37° C.
Bucket with ice
Micro scissors (FST)
Non-tissue-treated v-shaped 96-well plate (Sarstedt)

The production of astrocyte-containing Linkerspheres is as follows:

Human iPSC-derived astrocytes (AC) were differentiated according to Krencik et al. 2011 (Directed differentiation of functional astroglial subtypes from human pluripotent stem cells, Nat. Protoc. 6(11): 1710-7, doi:10.1038/nprot.2011.405). For each experiment, the astrocytes are thawed and cultured in ASC++ medium on 24- or 48-well plates. 6-7 days before the start of the experiment, the AC are treated with 1 ng/ml CNTF in ASC++ medium, with the medium being changed every other day. At least one full well is washed very carefully with PBS−− and incubated in TrypLE at 37° C. for 2 minutes to detach the AC. Once it is ensured that the cells are single cells, N2 medium in double the volume of the TrypLE-containing medium is added to stop the reaction. The cells are transferred to a 15-ml conical tube and centrifuged at 1500 g for 2 minutes. The supernatant is discarded, and the cells are resuspended in an appropriate volume of N2 medium to count the cells (about 500 μl-1000 μl) in a Neubauer chamber, diluted 1:1 with trypan blue to identify dead cells. The required number of cells is transferred to a 1.5 ml Eppendorf tube. To prepare one Astrolinker with a size of 2.5 μl per linker, 10,000 cells are needed.

The collected cells are then pelleted again for 2 minutes at 800 g. The supernatant is carefully and as completely as possible discarded. The cell pellet is then resuspended in cold BRDM medium to achieve 1.25 μl per astro linker (e.g., for 10 linkers/100,000 cells, 10 μl of medium is used). It is then stored on ice. Growth factor-reduced Matrigel (thawed overnight in the refrigerator) is added to the cooled cell suspension in a 1:1 ratio, and the solution is gently and thoroughly mixed, avoiding the formation of bubbles. To make the linkers more visible later, the Matrigel can be stained with ink or other dyes. Here, 5% of a 1:1000 dilution of ink in PBS was used.

Before beginning the procedure, 1.5 ml reaction tubes (1 tube per Astrolinker) are filled with ˜50 μl mineral oil and stored at room temperature until use.

2.5 μl of the astrocyte-Matrigel mixture is then transferred into the reaction tube filled with mineral oil using a thin 10 μl pipette tip (preferably pre-cooled). To create the linker, the pipette with its tip containing 2.5 μl astrocyte-Matrigel mix is held directly above the surface of the oil liquid and then pressed to the first stop. This forms a droplet at the front of the pipette. By dipping the pipette tip and the droplet into the oil liquid, the droplet detaches from the tip and sinks into the liquid. The remaining liquid in the pipette is discarded. The tube containing the linker is brought as quickly as possible to a heating block (37° C.) to allow for rapid solidification. This is necessary to prevent the cells from being unevenly distributed within the droplet. The tubes are then incubated at 37° C. for 15-30 minutes.

After incubation, about 200 μl of pre-warmed PBS (37° C.) is added. This is done so that the formed Linkersphere transitions from the oil phase to the aqueous phase. The Linkersphere plus the PBS (and as little oil as possible) are transferred into a petri dish (e.g., 6 cm) with pre-warmed PBS at 37° C. A cut 1000 μl tip is used to avoid damaging the Linkerspheres. This way, the Linkerspheres are washed, and the oil is removed. The washing steps can be repeated to remove oil residues. The washed Astrolinkers are transferred into a non-tissue-culture-treated 48-well plate containing 250 μl of pre-warmed ASC++ at 37° C. Again, cut tips are used. The Astrolinkers are cultured at 37° C., 20% O₂, and 5% CO₂ for at least overnight, and the medium is changed every 2-3 days (half medium change) until further use or fixation.

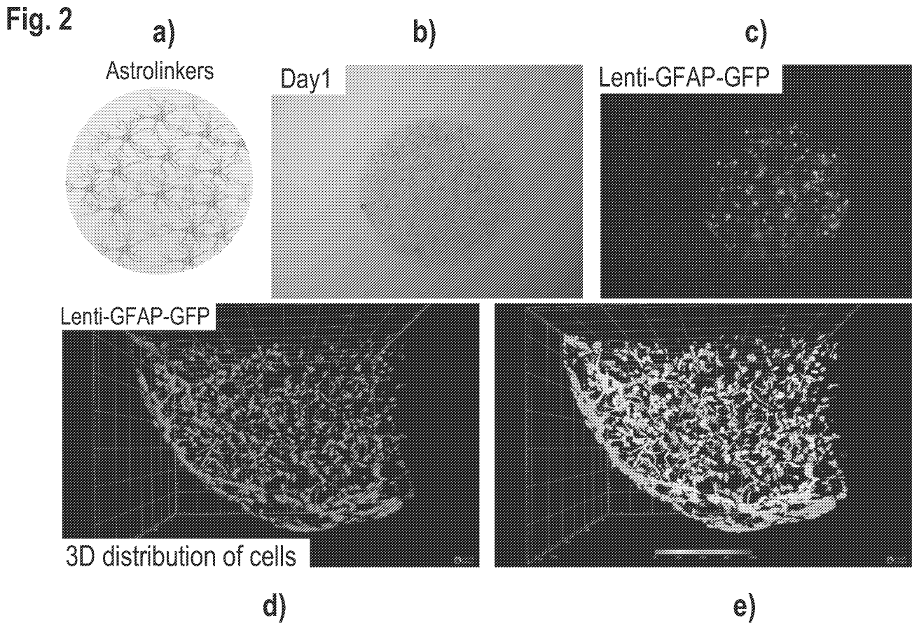

FIG. 2a schematically shows the Astrolinker. FIG. 2b shows an Astrolinker (linker sphere loaded with astrocytes) after one day in culture. FIG. 2c shows an Astrolinker with astrocytes that were previously transfected with a lentiviral construct (Lenti-GFAP-GFP) expressing a green fluorescent protein (GFP) under a glial fibrillary acidic protein (GFAP) promoter. FIGS. 2d and 2e show a three-dimensional reconstruction of a part of an Astrolinker, in which the astrocytes, as in 2c, express GFP under a GFAP promoter. FIG. 2d shows a fluorescence image stained in magenta, and FIG. 2e shows a height-coded false-color image.

Linkerspheres without Cells

The same protocol as described above is used for the production of cell-free linker spheres.

Instead of astrocytes, only medium (e.g., BRDM) is added to the GFR-Matrigel. All subsequent steps remain the same. Cell-free linker spheres can be cultured in any pre-warmed medium or buffer at 37° C.

### 2. Production of Double-Connections

Human iPSC-derived retinal organoids (RO), differentiated according to a previously published protocol (Zhong et al. 2014, Achberger et al. 2019), are selected after 40-80 days of differentiation.

On Day 1 (the day after linker production), before the connection, the ROs are cut into two parts using a micro-scissors and transferred into a non-tissue-culture-treated 96-well V-bottom plate. Each well receives one half of an organoid. Then, an Astrolinker or cell-free Linkersphere is added to the wells containing the ROs using a 1000 μl tip. Using a small needle or pipette tip, the ROs and the linker are positioned under a microscope. The RO is positioned so that the cut side directly touches the Astrolinker/Linkersphere. The plate is then very carefully placed in an incubator (37° C., 20% O₂, 5% CO₂) without disturbing the positioning.

The production of triple connections can optionally take place the following day or at any later time.

### 3. Production of Triple-Connections

One day later, thalamus organoids (TO), differentiated according to a previously published protocol (Xiang 2019), are selected after 40-80 days of differentiation. The individual TOs are added to the 96-well V-bottom plate containing the double-connection (Astrolinker/Linkersphere+RO). The triple-connection is made again using a small needle or tip, positioning the thalamus organoid directly on the opposite side of the retinal organoid attached to the Astrolinker/Linkersphere. This is crucial so that the cell connection must form through the Astrolinker and not directly. Without moving, the plate is stored overnight in the incubator at 37° C., 20% O₂, and 5% CO₂.

The cultivation of the triple-connection can optionally take place the following day.

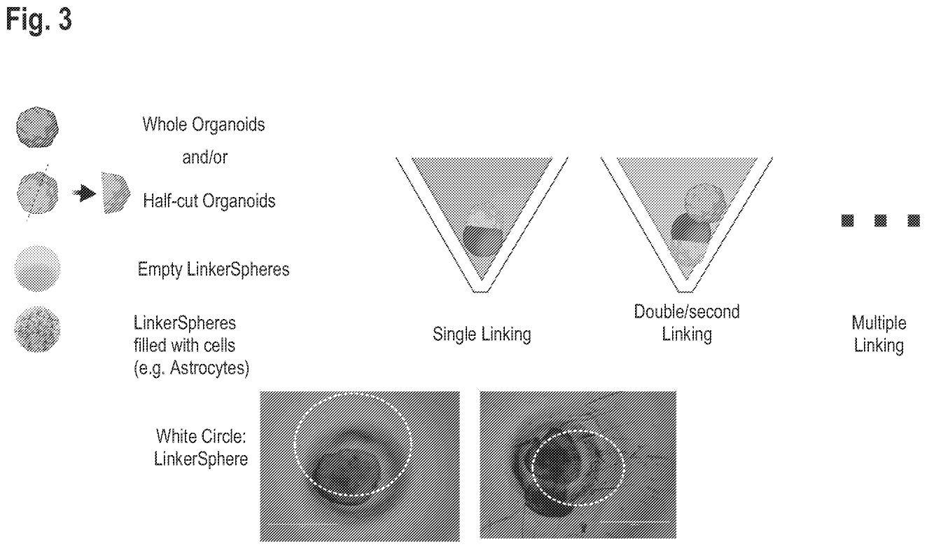

The connection process is schematically illustrated in FIG. 3. The scale bar in the two microscopic images shown at the bottom of the figure corresponds to a distance of 1000 μm.

### 4. Cultivation of Triple-Connections

On the next day, a non-tissue-culture-treated 48-well plate is prepared with 250 μl of pre-warmed BRDM medium at 37° C. The triple-connections are transferred into this 48-well plate (using cut 1000-μl tips) and incubated again at 37° C., 20% O₂, 5% CO₂. To observe the projections between RO and TO, the linkers are examined under a microscope. For this purpose, the RO can be stably transduced with GFP (e.g., using lentiviral vectors). The medium change for double or triple connections is carried out every second to third day with warm BRDM medium (250 μl per well, half medium change).

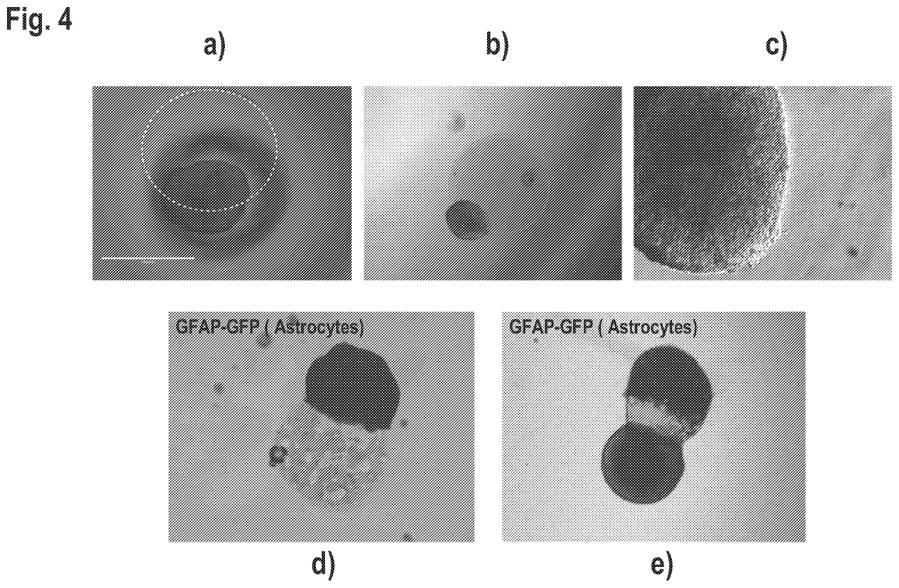

FIG. 4b shows the result of the connection process between a Linkersphere and a retinal organoid in a microscopic image. FIG. 4b shows the complex in a suspension culture. FIG. 4c shows the outgrowth of neurites from the organoid into the Linkersphere. FIG. 4d shows the result of a simple connection between a retinal organoid and a Linkersphere, while FIG. 4e shows the result of a double connection. The astrocytes are marked with GFP and accordingly stained.

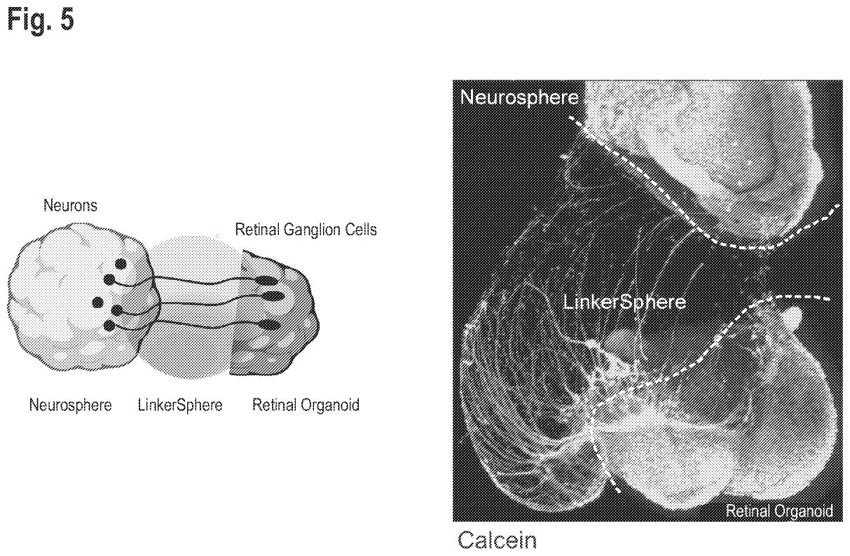

FIG. 5 shows a double connection of a Linkersphere with a Neurosphere on one side and a retinal organoid on the other side. The Neurosphere is connected to the retinal organoid via nerve pathways that extend through the Linkersphere. FIG. 6 shows that GFP-marked cells of the retinal organoid project into the interior of the Neurosphere and are positive for the ganglion cell marker NEFM.

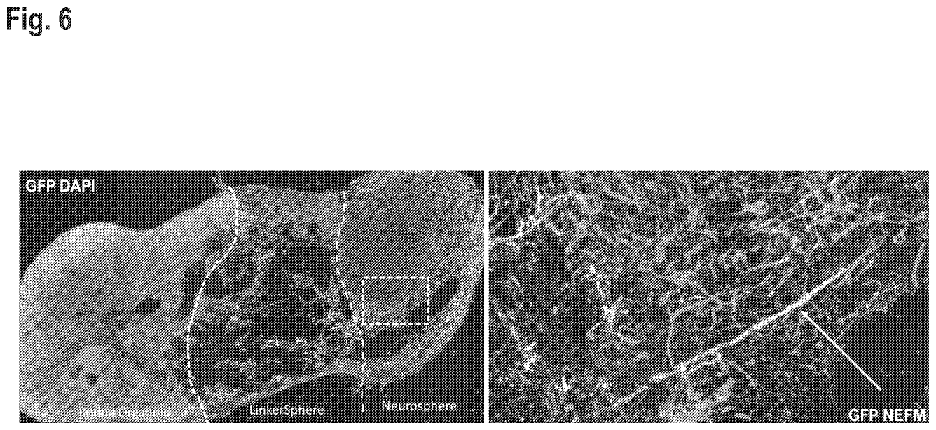

FIG. 7 shows a double connection of an Astrolinker with a Neurosphere on one side and a retinal organoid on the other side. The Neurosphere is connected to the retinal organoid via nerve pathways that extend through the Linkersphere.

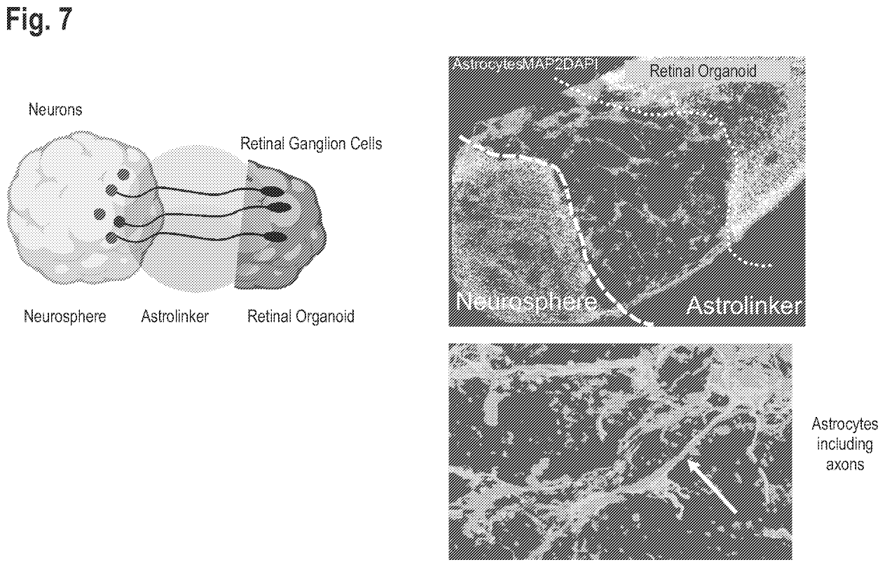

FIG. 8 schematically illustrates a complex optic nerve model. The complex optic nerve model, for example, for modeling glaucoma, consists of a retinal organoid containing retinal ganglion cells, two linkers filled with oligodendrocytes (myelinated part of the optic nerve) and astrocytes (intra-retinal part of the optic nerve), and brain organoids patterned, for example, for the diencephalon or metathalamus.

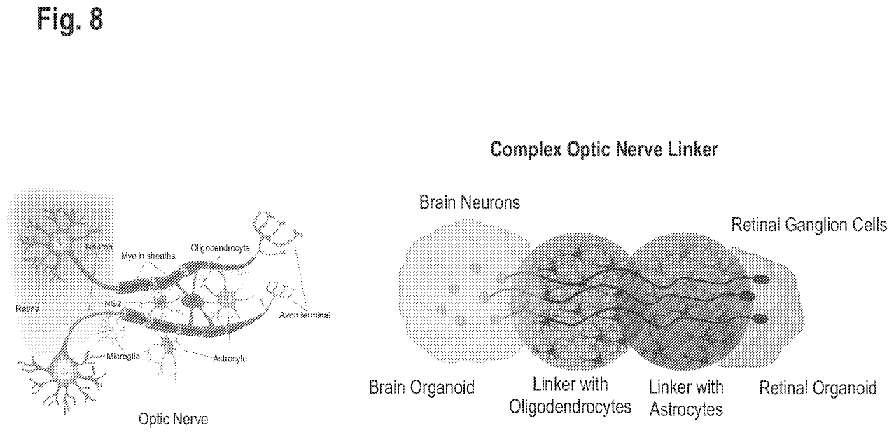

FIG. 9 schematically shows how vascularization can be supported using Linkerspheres, for example, by connecting tissue organoids with blood vessel organoids.

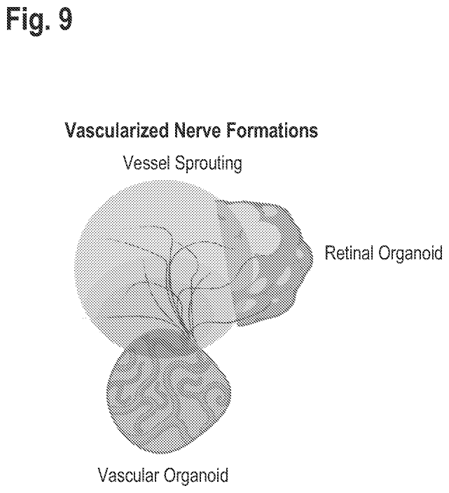

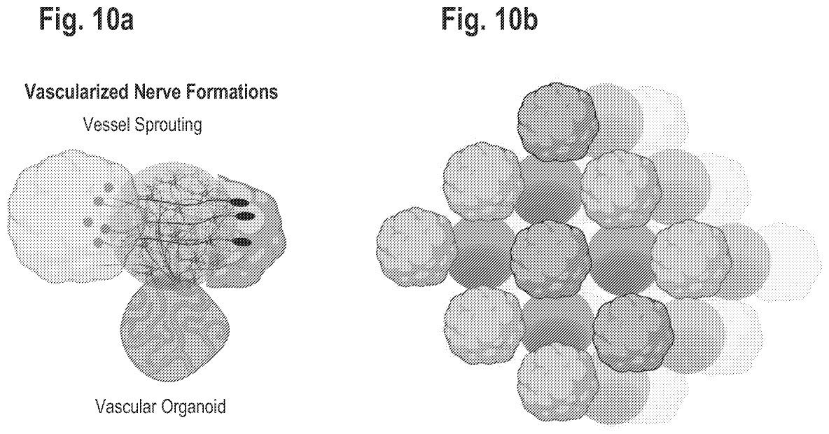

FIGS. 10A and 10B show multi-linking concepts. Linkerspheres allow for the combination of multiple organoids and various connective concepts (e.g., neuronal connections, vascularization). Linkerspheres could potentially facilitate the growth/assembly of complex multi-organoid and multicellular tissues, including nerve growth and vascularization.

1. A method for producing biocompatible structures for the connection and cultivation of biological material (“Linkerspheres”), comprising the following steps:
1) Providing a nonpolar, water-immiscible, biocompatible liquid;
2) Introducing a solution of a biocompatible matrix material into the liquid to obtain an emulsion;
3) Incubating the emulsion;
4) Forming a three-dimensional structure from the matrix material in the emulsion by incubation to obtain the Linkerspheres.

2. The method according to claim 1, wherein the nonpolar, water-immiscible, biocompatible liquid is mineral oil.

3. The method according to claim 1, wherein the matrix material is a basement membrane-like matrix.

4. The method according to claim 1, wherein in step (2) a solution of a growth factor-reduced basement membrane-like matrix is introduced.

5. The method according to claim 1, wherein in step (3) a treatment of the emulsion to solidify the matrix material takes place.

6. The method according to claim 5, wherein the treatment comprises exposure to heat.

7. The method according to claim 6, wherein the heat is at about 37° C.
The method according to claim 6, wherein the exposure is for about 15 to 30 minutes.

8. The method according to claim 1, wherein the solution of the matrix material comprises cell culture medium.

9. The method according to claim 1, wherein the solution of the matrix material comprises biological cells.

10. The method according to claim 1, wherein the solution of the matrix material comprises a dye.

11. The method according to claim 1, wherein in step (2) the solution of the matrix material is introduced into the mineral oil as fluid droplets.

12. The method according to claim 11, wherein the introduction of the solution into the mineral oil as fluid droplets takes place via a pipette tip.

13. The method according to one claim 1, wherein after step (4) the following step is carried out:
5) Isolating the Linkerspheres from the emulsion.

14. The method according to claim 13, wherein after step (5) the following step is carried out:
6) Washing the isolated Linkerspheres, preferably with an aqueous solution, more preferably with cell culture medium.

15. The method according to claim 12, wherein after step (4) and before step (5) the following step is carried out:
4′) Introducing an aqueous solution, preferably an aqueous buffer solution, into the emulsion to form an aqueous phase and transferring the Linkerspheres into the aqueous phase.

16. The method according to one of claim 13, wherein the isolated and optionally washed Linkerspheres are transferred into a culture vessel, preferably a culture dish.

17. The method according to claim 13, wherein the isolated and optionally washed and optionally transferred Linkerspheres are cultured.

18. The method according to claim 13, wherein the isolated and optionally washed and optionally transferred Linkerspheres are cultured at about 37° C.

19. The method according to claim 13, wherein the isolated and optionally washed and optionally transferred Linkerspheres are cultured at about 20 vol.-% O₂.

20. The method according to claim 13, wherein the isolated and optionally washed and optionally transferred Linkerspheres are cultured at about 5 vol.-% CO₂.

21. The method according to claim 13, wherein the isolated and optionally washed and optionally transferred Linkerspheres are cultured for at least about 12 hours.

22. The method for cultivating aggregates of biological material, comprising the following steps:
1) Bringing the aggregates into contact with biocompatible structures for the connection and cultivation of biological material (“Linkerspheres”) in a suitable medium to obtain a complex of the aggregates and the Linkerspheres,
2) Cultivating the complexes of the aggregates and the Linkerspheres,
wherein the Linkerspheres are obtained according to the method of claim 1.

23. The method according to claim 22, wherein the aggregates of biological material are cut before being brought into contact with the Linkerspheres, preferably with a microshear.

24. The method according to claim 23, wherein the aggregates are brought into contact with the Linkerspheres with the cut surface.

25. The method according to claim 22, wherein the cultivation takes place at about 37° C.

26. The method according to claim 22, wherein the cultivation takes place at about 20 vol.-% O₂.

27. The method according to claim 22, wherein the cultivation takes place at about 5 vol.-% CO₂.

28. The method according to claim 22, wherein the aggregates of biological material are organoids and/or spheroids or assembloids.

29. The method according to claim 28, wherein the aggregates of biological material are selected from the group consisting of: retinal organoid, vascular organoid, brain organoid, neurospheroid.

30. The method according to claim 22, wherein the aggregates of biological material comprise astrocytes.

31. The method according to claim 22, wherein the aggregates of biological material comprise astrocytes derived from induced pluripotent stem cells (iPSCs).

32. The method according to claim 16, wherein after step (2) the following step is carried out:
(3) Repeating steps (1) and (2).
